(19) United States
(12) Patent Application Publication
(10) Pub. No.: US 2026/0297495 A1
(43) Pub. Date: Oct. 1, 2026

(54) METHOD FOR WET-MILLING AND RELATED CONTROL SYSTEM

(71) Applicant: Novozymes A/S, Bagsvaerd (DK)

(72) Inventors: Gregory Michael AMES, Copenhagen (DK); Scott Robert MCLAUGHLIN, Wake Forest, NC (US); Thomas Erik NILSSON, Virum (DK); Prachand SHRESTHA, Morrisville, NC (US)

(73) Assignee: Novozymes A/S, Bagsvaerd (DK)

(21) Appl. No.: 18/877,487

(22) PCT Filed: Jul. 3, 2023
(86) PCT No.: PCT/US2023/069569

§ 371 Date: Dec. 20, 2024

Related U.S. Application Data
(60) Provisional application No. 63/358,299, filed on Jul. 5, 2022.

(51) Int. Cl. C12M 1/36; C12M 1/00; C12M 1/40; C12P 19/04; C12P 21/00

(52) U.S. Cl. CPC C12M 41/48; C12M 21/18; C12M 41/14; C12P 19/04; C12P 21/00

(57) ABSTRACT

A control system for a fiber washing system of a wet milling system and related methods are disclosed, the control system comprising a controller comprising one or more processors and an interface, wherein the one or more processors are configured to: obtain fiber data comprising an input fiber parameter indicative of a fiber input to an incubation tank of the fiber washing system; obtain a time parameter indicative of an incubation time for slurry in the incubation tank; determine an input scheme based on the fiber data and the time parameter; and control one or more input devices of the wet milling system according to the input scheme.

The present disclosure relates to wet-milling and in particular to fiberwashing in wet-milling and control thereof. In particular, a control system for a fiberwashing system of a wet-milling system, and a method of producing a fiber output from a fiberwashing system of a wet-milling system is disclosed.

### BACKGROUND

Grain wet-milling and processes associated therewith receives increased interest in efforts to increase starch and protein/gluten yields and reduce waste. Today, most of the starch and protein present in corn kernels are separated and enriched into respective co-product streams via series of mechanical and separation stages in a typical mechanical corn wet-milling (CWM) process. However, some starch and protein remains bound to corn fiber matrix of corn kernels. WO 2018/053220 relates to an improved wet-milling process, wherein an enzyme incubation tank is integrated as part of a fiber-washing system, thus increasing the yield of starch and protein bound to the fiber fraction. These yield results are dependent on incoming fiber parameters (for example, fiber solids, bound starch and protein values), incubation time, temperature, pH and most importantly dosage of enzyme. While incubation time, temperature and pH values are routinely monitored and adjusted in plant operation, the enzyme dosing is not, and dosing is based on volume of corn kernel being steeped and ground in initial stages of the process, which precedes the enzymatic Fiber-washing step by 24 to 48 hours. Furthermore, fiber solids are not monitored and this has huge fluctuations in the fiber slurry heading into incubation tank. Therefore, the current process has disparity in adequate enzyme dose to fiber solids directed to incubation tank. This practice impacts enzyme efficiency and leads to major variation in quantities of starch and protein yields specifically due to over or under dosing of enzymes required to keep consistent yield targets. Thus, there is a need in the industry to improve the fiber-washing step in order to avoid under- and over-dosing of enzymes.

### SUMMARY

Accordingly, there is a need for methods and devices optimizing yield and in particular starch and/or protein yield in wet-milling and processes related to wet-milling.

A control system for a fiberwashing system of a wet-milling system or parts thereof is disclosed, the control system comprising a controller comprising one or more processors and an interface. The one or more processors are configured to obtain, e.g. via the interface, fiber data, e.g. comprising an input fiber parameter indicative of a fiber input to an incubation tank of the fiberwashing system; obtain, e.g. via the interface and/or using the one or more processors, a time parameter indicative of an incubation time for slurry in the incubation tank; determine, e.g. using the one or more processors, an input scheme based on the fiber data and the time parameter; and control, e.g. via the interface and/or using the one or more processors, one or more input devices of the wet-milling system according to the input scheme.

Further, a fiberwashing system is disclosed, the fiberwashing system comprising a control system as described herein. The fiberwashing system comprises an incubation tank and a plurality of screening units, such as in the range from 2 to 10 screening units, including a first screening unit and a second screening unit downstream of the first screening unit, the first screening unit having a first fiber input and a first fiber output, and the second screening unit having a second fiber input and a second fiber output, wherein a first fiber output of the first screening unit or a second fiber output of the second screening unit is optionally coupled to an incubation input of the incubation tank.

Further, an enzymatic corn wet-milling process is disclosed, the enzymatic corn wet-milling process comprising using the control system as described herein for fiberwashing in the wet-milling process.

Also, a use of a control system as described herein in a corn wet-milling process is disclosed, e.g, wherein the control system is applied to an enzyme incubation tank integrated into a fiber-washing system.

A computer-implemented method of producing a fiber output from a fiberwashing system of a wet-milling system is disclosed, the method comprising obtaining fiber data comprising an input fiber parameter, e.g. indicative of a fiber input to an incubation tank of the fiberwashing system; obtaining a time parameter, e.g. indicative of an incubation time for slurry in the incubation tank; determining an input scheme based on the fiber data and/or the time parameter; and controlling one or more input devices of the wet-milling system according to the input scheme.

The present disclosure allows optimization of fiberwashing and input to a wet-milling system which in turn may lead to increased or optimized yield of starch and/or protein. Further, an increased fiber purity of the fiber slurry being output from the fiber-washing system may be provided.

It is an important advantage of the present disclosure that a more accurate and improved fiberwashing control is provided which in turn allows for a more stable and optimal wet-milling process, e.g. to achieve increased corn or kernel component separation. In particular, the real-time monitoring and determination of the fiber input and/or fiber output allows more precise control of the fiberwashing process.

Further, the use of in situ real-time online monitoring and control of the wet-milling system reduces the risk of underdosing which may lead to reduced starch/protein yield.

### BRIEF DESCRIPTION OF THE DRAWINGS

The above and other features and advantages of the present invention will become readily apparent to those skilled in the art by the following detailed description of exemplary embodiments thereof with reference to the attached drawings, in which:

FIG. 1 schematically illustrates an exemplary wet-milling system according to the present disclosure,

FIG. 2 is a block diagram of example parts of an example fiberwashing system,

FIG. 3 is a block diagram of example parts of an example fiberwashing system, and

FIG. 4 is a flow chart of an exemplary method of producing a fiber output from a fiberwashing system of a wet-milling system,

FIG. 5 is a flow chart of an exemplary method of producing a fiber output,

FIG. 6 shows example starch release profiles,

FIG. 7 shows example protein release profiles,

FIG. 8 illustrates an enzyme performance model for protein yield, and

FIG. 9 illustrates an enzyme performance model for protein yield.

FIG. 10 illustrates how enzyme dosing is readjusted based on real-time input and output parameters (collected by instruments and sensor) for optimal performance. These parameters may comprise of fiber solid content and density data of the flow heading to incubation tank, temperature data, tank fill levels and MIR spectral data from the sensors/probes. The optimal target parameters are protein and starch yields from incoming fiber solids and flow, after fiber-enzyme interactions in the incubation tank. The enzymatic Fiberwashing process is optimized based on real-time inline measurement system data and calibrated enzyme dose.

FIG. 11 shows effect of five different enzyme dosing schemes and three different fiber solid contents, on the release of insoluble protein bound in fiber over time. It can be seen that protein in fiber (PIF) is reduced. For example, to reduce the fiber protein by 1% (i.e., for target PIF to be at 5.8%) it requires 0.35 kg enzyme/ton corn if the fiber solid is 4%. For same target PIF at 5.8%, it requires 0.2 and 0.17 kg enzyme/ton corn respectively if the fiber solids are 5% and 6%.

FIG. 12 shows an example of Mid infrared (MIR) spectral data recorded by a MIR sensor/probe. The spectrometer probe collects real-time data in the reaction vessel comprising corn fiber solids and enzyme products. The changes in spectral values (absorbance) across various wavenumbers is linked with activity of enzymes on corn fiber between two time points, for example: T=0 and T=180 minutes for 4.5% corn fiber solids and enzyme dose of 0.25 kg enzyme/ton corn.

FIG. 13 shows enzymatic hydrolysis of corn fiber with an enzyme blend (cellulases and GH5 xylanase) and the resulting release of bound starch and protein from corn fiber. Thereby, residual starch in fiber (SIF) and protein in fiber (PIF) were reduced after 180 minutes of incubation time. The enzyme was dosed at 0.25 kg/ton corn for 4.5% corn fiber solid at pH 4.4, temperature 48 degree Celsius.

FIG. 14 shows enzymatic hydrolysis of corn fiber with an enzyme blend (cellulase and GH5 xylanase) and the resulting release of bound starch and protein from corn fiber. Therefore, residual starch in fiber (SIF) and protein in fiber (PIF) were reduced while concentration of the corn fiber hydrolysis products (i.e., different sugars) increased over 150 minutes of incubation time. The enzyme was dosed at 0.30 kg/ton corn for 5% corn fiber solid at pH 4.4, temperature 48 degree Celsius.

### DETAILED DESCRIPTION

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

A control system for a wet-milling system, such as a control system for a fiberwashing system of a wet-milling system is disclosed. The control system comprises a controller comprising one or more processors and an interface.

In one or more examples, a control system for a fiberwashing system of a wet-milling system is disclosed, the control system comprising a controller comprising one or more processors and an interface, wherein the one or more processors are configured to obtain fiber data comprising an input fiber parameter indicative of a fiber input to an incubation tank of the fiberwashing system; obtain a time parameter indicative of an incubation time for slurry in the incubation tank; determine an input scheme based on the fiber data and the time parameter; and control one or more input devices of the wet-milling system according to the input scheme.

The one or more processors of the controller are configured to obtain, such as one or more of determine, measure, receive, and retrieve, fiber data, e.g. via the interface. The fiber data may be indicative of one or more fiber input and/or one or more fiber output, such as fiber content and/or fiber/slurry flows in the wet-milling system. The wet-milling system may be a corn wet-milling system.

The fiber data denoted FD comprises one or more input fiber parameters denoted IFP_1, IFP_2, . . . IFP_N, indicative of a fiber input to an incubation tank of the fiberwashing system. The input fiber parameter(s) indicative of a fiber input to an incubation tank of the fiberwashing system may be obtained based on input fiber data/sensor data also denoted ISD from one or more sensors arranged in the wet-milling system, such as at an input of the incubation tank.

For example, the fiber data may comprise, as a first input fiber parameter IFP_1, a fiber percentage indicative of fiber content in a slurry, e.g. mass or volume of fiber/mass or volume of slurry, and/or, as a second input fiber parameter, a slurry flow, e.g. volume of slurry/time. In other words, the first input fiber parameter IFP_1 may be a fiber percentage in a slurry and/or the second input fiber parameter IFP_2 may be a slurry flow of slurry into the incubation tank.

The one or more processors of the controller are configured to determine an input scheme based on the fiber data, such as IFP_1 and/or IFP_2, and the time parameter. The input scheme may comprise and define control parameters for one or more input devices in the wet-milling system, e.g. for one or more pump devices, one or more valves, one or more conveyors or other device(s) operating as input devices in the wet-milling system.

The one or more processors of the controller are configured to control one or more input devices of the wet-milling system according to the input scheme. For example, to control one or more input devices of the wet-milling system may comprise to output or transmit, e.g. via the interface, one or more control parameters also denoted input control parameters (ICPs) including one or more enzyme control parameters (ECPs) to input device(s) of the wet milling system.

In one or more examples, the fiber data comprises spectrometer data and/or density data of the fiber input, and wherein to obtain fiber data comprises to determine one or more parameters, such as input fiber parameter(s) and/or output fiber parameter(s) based on the spectrometer data and/or density data. The spectrometer data may be obtained from a MIR (Mid Infra-Red) and/or NIR (Near Infra-Red) sensor (probe) device, such as Keit instrument. The density data may be obtained from a density meter, such as a Coriolis meter.

In one or more examples, the slurry flow in and/or out of the incubation tank is measured with a Coriolis meter or magnetic flow meter, e.g. forming a part of the input sensor device and/or the output sensor. Advantageously, a Coriolis meter also provides density and/or a mass flow.

In one or more examples, the one or more input fiber parameters is indicative of a fiber content in a slurry input to the incubation tank.

The fiber data may comprise one or more output fiber parameters denoted OFP_1, OFP_2, . . . , OFP_M indicative of a fiber output from the incubation tank of the fiberwashing system. The output fiber parameter(s) indicative of a fiber output from the incubation tank of the fiberwashing system may be obtained based on output fiber data/sensor data also denoted OSD from one or more sensors arranged in the wet-milling system, such as at an output of the incubation tank.

The output fiber parameter(s) may be indicative of a fiber content in a slurry output from the incubation tank. For example, the fiber data may comprise, as a first output fiber parameter OFP_1, a fiber percentage indicative of fiber content in a slurry, e.g. mass or volume of fiber/mass or volume of slurry, and/or, as a second output fiber parameter, a slurry flow, e.g. volume of slurry/time. In other words, the first output fiber parameter OFP_1 may be a fiber percentage in a slurry and/or the second output fiber parameter OFP_2 may be a slurry flow of slurry out of the incubation tank.

In one or more examples, to obtain a time parameter denoted t_res indicative of an incubation time for slurry in the incubation tank comprises to obtain a tank level parameter, such as current slurry level from one or more sensors, and to determine the time parameter based on the tank level parameter. The tank level parameter may be slurry level in percentage of the incubation tank volume, slurry level by volume, or slurry level by mass.

In one or more examples, to obtain a time parameter indicative of an incubation time for slurry in the incubation tank comprises to obtain a tank input flow parameter TIFP, e.g. with input flow meter of input sensor device, and to determine the time parameter based on the tank input flow parameter. In other words, the time parameter t_inc may be a function of the tank input flow parameter TIPF.

In one or more examples, to obtain a time parameter indicative of an incubation time for slurry in the incubation tank comprises to obtain a tank output flow parameter TOFP, e.g. with output flow meter of output sensor device, and to determine the time parameter based on the tank output flow parameter. In other words, the time parameter t_inc may be a function of the tank output flow parameter TOPF optionally in combination with TIPF.

In one or more examples, the time parameter denoted t_inc indicative of an incubation time for slurry in the incubation tank may be given as:

$$t\_inc = V\_slurry / TIFP ,$$

where V_slurry is the slurry level of slurry in the incubation tank and TIFP is a tank input flow parameter indicative of the flow rate of fiber slurry into the incubation tank, e.g. based on input sensor data ISD from input sensor device e.g. comprising an input flow meter. The slurry level V_slurry may be based on the tank volume and a current tank fill level (in percent), e.g. based on level data LD from a level sensor of tank sensor device and/or be obtained from system control.

In one or more examples, the fiber data FD comprises a constituent input parameter denoted CIP indicative of a quantity of insoluble protein and starch entering the incubation tank, and wherein to determine an input scheme based on the fiber data and the time parameter comprises to determine the input scheme based on the constituent input parameter. In other words, the input scheme may be a function of CIP.

In one or more examples, the fiber data comprises a protein input parameter indicative of a quantity of insoluble protein entering the incubation tank, and wherein to determine an input scheme based on the fiber data and the time parameter comprises to determine the input scheme based on the protein input parameter.

In one or more examples, the fiber data comprises a starch input parameter indicative of a quantity of insoluble starch entering the incubation tank, and wherein to determine an input scheme based on the fiber data and the time parameter comprises to determine the input scheme based on the starch input parameter.

In one or more examples, the one or more processors are configured to obtain a protein output parameter indicative of a quantity of insoluble protein exiting or output from the incubation tank, and wherein to determine an input scheme based on the fiber data and the time parameter comprises to determine the input scheme based on the protein output parameter.

The input scheme may comprise an enzyme dosing scheme. The enzyme dosing scheme is indicative of and defines enzyme control parameter(s) also denoted ECP_1, ECP_2, . . . for controlling enzyme dosing in the wet-milling system.

In one or more examples, to determine an input scheme comprises to determine an enzyme dosing scheme based on the fiber data and the time parameter, the enzyme dosing scheme comprising a first enzyme flowrate or at least a first enzyme control parameter for a first enzyme, and wherein to control one or more input devices comprises to control a first enzyme input device according to the enzyme dosing scheme, such as a first enzyme flowrate or first enzyme amount. In other words, the enzyme dosing scheme may comprise a first enzyme control parameter ECP_1, such as a first enzyme flowrate and/or a first enzyme amount or indicative thereof. The first enzyme may be a first enzyme or a first enzyme composition.

In one or more examples, to control an input device comprises to control the first enzyme input device according to the first enzyme flowrate and/or according to other first enzyme control parameter(s), such as a first enzyme distribution and/or the first enzyme amount. In other words, the input scheme may comprise an enzyme dosing scheme comprising a first enzyme control parameter, and the controller may be configured to output or transmit a first enzyme control parameter ECP_1 indicative of or being the first enzyme flowrate and/or the first enzyme amount, e.g. to the first enzyme input device, such as a dosing pump or valve. The first enzyme input device may be a metering pump or a gravity flow dispenser having a controllable outflow valve configured for controlling the amount of enzyme flowing through the flow valve and into the incubation tank.

In one or more examples, the first enzyme is selected from a xylanase and a cellulase or is a first enzyme composition comprising one or more of a xylanase and a cellulase.

In one or more examples, the xylanase belongs to the GH family 5, 8, 10, 11, or 30, and comprises one or more of GH5 xylanase, GH8 xylanase, GH30 xylanase, GH10 xylanase, and GH11 xylanase.

In one or more examples, the first enzyme is or comprises GH62 arabinofuranosidase.

In a preferred embodiment the xylanase is a GH5_21 xylanase (Xylanase A) derived from Chryseobacterium sp-10696 and disclosed in WO 2021/122867. In another preferred embodiment the xylanase is a GH10 xylanase and preferably used in combination with a GH62 arabinofuranosidase.

In one or more examples, the cellulase comprises one or more of endoglucanase, cellobiohydrolase, and beta-glucosidase. In a preferred embodiment the cellulases are a whole cellulase (Cellulase A) derived from Trichoderma reesei. This cellulase composition will comprise all cellulase activities expressed in T. reesei.

In one or more examples, the enzyme dosing scheme comprises a second enzyme flowrate or at least a second enzyme control parameter for a second enzyme, and wherein to control an input device comprises to control a second enzyme input device according to the second enzyme flowrate or a second enzyme amount. In other words, the enzyme dosing scheme may comprise a second enzyme control parameter ECP_2, such as a second enzyme flowrate and/or a second enzyme amount or indicative thereof. The second enzyme may be a second enzyme or a second enzyme composition. The second enzyme may be different from the first enzyme. In other words, the controller may be configured to determine and control input of a plurality of enzymes or enzyme compositions, thereby allowing tailoring the input on enzymes to the fiber input.

In one or more example controllers, to control an input device comprises to control the second enzyme input device according to the second enzyme control parameter, such as the second enzyme flowrate and/or the second enzyme amount. In other words, the input scheme may comprise an enzyme dosing scheme comprising a second enzyme control parameter, and the controller may be configured to output or transmit a second enzyme control parameter ECP_2 indicative of or being the second enzyme flowrate and/or the second enzyme amount, e.g. to the second enzyme input device, such as a dosing pump or valve. The second enzyme input device may be a metering pump or a gravity flow dispenser having a controllable outflow valve configured for controlling the amount of enzyme flowing through the flow valve and into the incubation tank.

The enzyme dosing scheme may define a rate or a difference between different enzyme flowrates. For example, the enzyme dosing scheme may comprise a first ratio indicative of ratio between a first enzyme flowrate and a second enzyme flowrate. In other words, to control an input device may comprise to control a ratio between input of the first enzyme and input of the second enzyme.

In one or more examples, to determine an input scheme comprises to determine a feed rate, and wherein to control one or more input devices comprises to control a feed input device according to the feed rate. The feed rate may comprise a process water feed rate or other water source feed rate. The feed rate may comprise a mill feed rate.

In one or more examples, the fiber data/sensor data comprises spectrometer data and/or density data of the fiber input. To obtain fiber data optionally comprises to determine a first component parameter of a first component of the fiber input based on the spectrometer data and/or density data. The spectrometer data may be obtained from a MIR (Mid Infra-Red) and/or NIR (Near Infra-Red) sensor device, such as Keit instrument. The density data may be obtained from a density meter, such as a Coriolis meter. The input scheme, such as enzyme dosing scheme, may be based on the first component parameter also denoted CP_1.

In one or more examples, the first component is one of a starch, a protein, and fiber.

In one or more examples, to obtain fiber data comprises to determine a second component parameter of a second component or second component composition of the fiber input based on the spectrometer data. The second component is different from the first component and may be one of a starch, a protein, and fiber. The second component may be a combination and/or constituents of two or more of starch, protein and fiber. The input scheme, such as enzyme dosing scheme, may be based on the second component parameter also denoted CP_2.

In one or more examples, the fiber data comprises temperature data indicative of a temperature of the fiber input, and wherein to determine an input scheme based on the fiber data and the time parameter comprises to determine the input scheme, such as enzyme dosing scheme, based on the temperature data.

In one or more examples, the fiber data comprises pH data indicative of a pH of the fiber input, and wherein to determine an input scheme based on the fiber data and the time parameter comprises to determine the input scheme, such as enzyme dosing scheme, based on the pH data.

A fiberwashing system is disclosed. The fiberwashing system comprises a control system; optionally an incubation tank; and a plurality of screening units including a first screening unit and a second screening unit downstream the first screening unit. The incubation tank may be integrated into the fiberwashing system. In one or more example fiberwashing systems, the number of screening units in the fiberwashing system is in the range from 4 to 10, the screening units being arranged between a fiber input and a fiber output of the fiberwashing system. The retention time is the time it takes for fiber slurry to pass through the fiberwashing system from the fiber input to the fiber output and is also denoted T_retention. In one or more example fiberwashing systems, the retention time T_retention is in the range from 1 to 5 hours, such as in the range from 1.5 hours to 4 hours.

The incubation tank has a incubation input and an incubation output. The incubation time is the time it takes for fiber slurry to pass through the incubation tank from the incubation input to the incubation output and is also denoted t_inc. In one or more example fiberwashing systems, the incubation time t_inc is in the range from 0.5 hour to 3 hours.

In one or more examples, the first screening unit has a first fiber input and a first fiber output, and the second screening unit has a second fiber input and a second fiber output, wherein a first fiber output of the first screening unit or a second fiber output of the second screening unit is coupled, such as directly coupled, to an incubation input of the incubation tank.

In one or more examples, the second fiber input of the second screening unit is coupled, such as directly coupled, to an incubation output of the incubation tank. In other words, slurry from the incubation tank may be fed, e.g. directly fed into the second screening unit.

The incubation tank is configured for enzyme incubation and may be arranged in the fiberwashing system where the fiber content also denoted dry solid fiber content has increased to about 4-10% (w/w) in order to obtain optimal reaction conditions for enzyme hydrolysis. When the slurry also denoted process stream enters fiber input of the fiberwashing system, the fiber content is typically in the range from 1.5-2.0% (w/w) and since there will be an up-concentration of the fiber material in the slurry as the slurry moves through the screening units/stages of the fiberwashing system, the optimal positioning of the incubation tank will be somewhere between middle or intermediate stages/screening units of the fiberwashing system. For example, e.g. in a fiberwashing system with 6-8 screening units/stages, the incubation tank may be arranged between the screening unit of the third stage and the screening unit of the fourth stage, between the screening unit of the fourth stage and the screening unit of the fifth stage, or between the screening unit of the fifth stage and the screening unit of the sixth stage. In one or more examples, the incubation tank is arranged before one, two, or at least three screening units, i.e. one, two, or at least three screening units are arranged between the incubation tank and the fiber output of the fiberwashing system. Arranging the incubation tank between intermediate screen units/stages of the fiberwashing system may allow sufficient up-concentration of fiber material in the slurry and at the same time allow sufficient washing of enzyme-incubated slurry.

In one or more examples, the incubation tank is arranged between the screening unit of a second stage and the screening unit of a third stage of the fiberwashing system. In other words, e.g. for a fiberwashing system having in the range from 6 to 10 screening units, a fiber output of a second stage screening unit may be coupled to the incubation input of the incubation tank and a fiber input of a third stage screening unit may be coupled to the incubation output of the incubation tank.

In one or more examples, the incubation tank is arranged between the screening unit of a third stage and the screening unit of a fourth stage of the fiberwashing system. In other words, e.g. for a fiberwashing system having in the range from 6 to 10 screening units, a fiber output of a third stage screening unit may be coupled to the incubation input of the incubation tank and a fiber input of a fourth stage screening unit may be coupled to the incubation output of the incubation tank.

In one or more examples, the incubation tank is arranged between the screening unit of a fourth stage and the screening unit of a fifth stage of the fiberwashing system. In other words, e.g. for a fiberwashing system having in the range from 6 to 10 screening units, a fiber output of a fourth stage screening unit may be coupled to the incubation input of the incubation tank and a fiber input of a fifth stage screening unit may be coupled to the incubation output of the incubation tank.

The first screening unit may be arranged in a first stage of the fiberwashing system and the second screening unit may be arranged in a second stage of the fiberwashing system. The first screening unit and the second screen unit may be neighbouring screen units.

The first screening unit may be arranged in a second stage of the fiberwashing system, i.e. another screening unit is arranged upstream the first screening unit in a first stage of the fiber washing system, and the second screening unit may be arranged in a third stage of the fiberwashing system.

The first screening unit may be arranged in a third stage of the fiberwashing system, i.e. two screening units are arranged upstream the first screening unit in the fiber washing system, and the second screening unit may be arranged in a fourth stage of the fiberwashing system.

The first screening unit may be arranged in a fourth stage of the fiberwashing system, i.e. three screening units are arranged upstream the first screening unit in the fiber washing system, and the second screening unit may be arranged in a fifth stage of the fiberwashing system.

The first screening unit may be arranged in a fifth stage of the fiberwashing system, i.e. four screening units are arranged upstream the first screening unit in the fiber washing system, and the second screening unit may be arranged in a sixth stage of the fiberwashing system.

A wet-milling process is disclosed, wherein the wet-milling process comprises using the control system as described herein for fiberwashing in the wet-milling process. The wet-milling process may be an enzymatic corn wet-milling process.

In one or more examples, starch yield and/or corn gluten yield from ground corn kernels is increased compared to an enzymatic corn wet-milling process where no control system is in place.

In one or more examples, the wet-milling process comprises a) soaking the corn kernels in water to produce soaked kernels; b) grinding the soaked kernels to produce ground kernels; c) separating germ from the ground kernels to produce a corn kernel mass comprising fiber, starch and gluten; and d) subjecting the corn kernel mass, particularly a ground fiber fraction of the corn kernel mass, to a fiberwashing procedure separating starch and gluten from the fiber to produce a fiber output, wherein at least one xylanase and/or one or more cellulases are present/added before or during step d), and wherein the control system is applied to an enzyme incubation tank in the fiber-washing system in step d).

In one or more examples, the wet-milling process comprises e) separating the starch from the gluten; and f) washing the starch.

In one or more examples, a computer-implemented method of producing a fiber output from a fiberwashing system of a wet-milling system is disclosed, the method comprising obtaining fiber data comprising an input fiber parameter indicative of a fiber input to an incubation tank of the fiberwashing system; obtaining a time parameter indicative of an incubation time for slurry in the incubation tank; determining an input scheme based on the fiber data and the time parameter; and controlling one or more input devices of the wet-milling system according to the input scheme.

The control system optionally comprises a sensor system. The sensor system comprises one or more sensors connected to controller(s) of the control system for provision of sensor data to the controller. The control system may be a distributed control system. In other words, the control system may comprise a plurality of controllers, each controller implementing one or more control schemes to control the wet-milling system or parts thereof, such as the fiberwashing system of the wet-milling system.

In one or more examples, such as example controllers, to determine an input scheme comprises to determine a feed rate and/or a feed scheme, e.g. of one or more of water, corn, corn kernels, and corn kennel mass, and wherein to control one or more input devices optionally comprises to control a feeder input device according to the feed rate/feed scheme. The feed scheme optionally comprises a first feed rate or first feed amount for a first feeder input device, e.g. for feeding corn kernel mass to the fiberwashing system. To control one or more input devices optionally comprises to control a first feeder input device according to the feed rate/feed scheme, such as a first feed rate or first feed amount. In other words, the feed scheme may comprise a first feed control parameter FCP_1, such as a first feed rate and/or a first feed amount or indicative thereof.

In one or more examples, such as example controllers, to control an input device comprises to control the first feeder input device according to the first feed control parameter, such as (first) feed rate and/or the (first) feed amount. In other words, the input scheme may comprise a feed rate and/or a feed scheme comprising a first feed control parameter, and the controller may be configured to output or transmit a first feed control parameter FCP_1 indicative of or being the first feed rate and/or the first feed amount, e.g. to the first feeder input device.

In one or more examples, such as example controllers, to determine a feed scheme comprises to determine a second feed rate or second feed amount for a second feeder input device, e.g. for feeding one or more of water, corn, corn kernels, and corn kennel mass to the fiberwashing system, and wherein to control one or more input devices optionally comprises to control a second feeder input device according to the feed scheme, such as the second feed rate or second feed amount for a second feeder input device, e.g. for feeding grain or other raw material to the bioethanol system. In other words, the feed scheme may comprise a second feed control parameter FCP_2, such as a second feed rate and/or a second feed amount or indicative thereof.

In one or more examples, such as example controllers, to control an input device comprises to control the second feeder input device according to the second feed control parameter, such as second feed rate and/or the second feed amount. In other words, the input scheme may comprise a feed rate and/or a feed scheme comprising a second feed control parameter, and the controller may be configured to output or transmit a second feed control parameter FCP_2 indicative of or being the second feed rate and/or the second feed amount, e.g. to the second feeder input device.

The control system may comprise one or more sensors including a first sensor and/or a second sensor. The sensor(s) provide sensor data for the controller(s), e.g. for determining the input scheme based on the sensor data. The sensor data may form at least a part of the fiber data and/or be used for determining fiber data, such as input and/or output fiber parameters.

In one or more example control systems, the control system, such as the one or more sensors, comprises a density meter, such as a Coriolis meter, and/or a spectrometer, such as a Keit instrument.

In one or more example control systems, the control system comprises a thermometer (second sensor or second sensor device) for provision of temperature data indicative of a temperature of the fiber input.

In one or more example control systems, the control system comprises a pH meter (third sensor or third sensor device) for provision of pH data indicative of a pH of the fiber input,

In one or more example controllers, to determine a first enzyme flowrate or a first enzyme amount for a first enzyme is based on the fiber data, such as one or more input fiber parameters and/or one or more output fiber parameters.

In one or more examples, such as example controllers, to determine a first enzyme flowrate or a first enzyme amount for a first enzyme is based on the first component parameter and/or one or more other component parameters, such as the second component parameter.

In one or more examples, such as example controllers, to determine a second enzyme flowrate or a second enzyme amount for a second enzyme is based on the second component parameter and/or one or more other component parameters, such as the first component parameter.

It is noted that descriptions and features of control system, fiberwashing system, controller, and functionality thereof also applies to methods and vice versa.

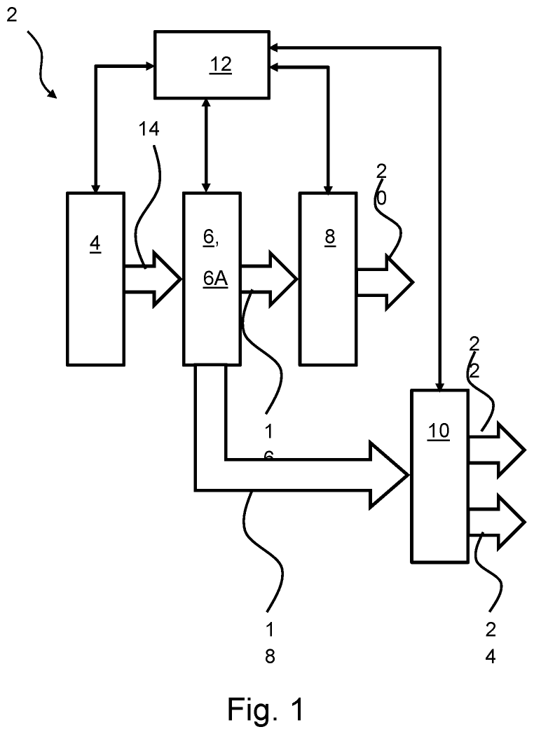

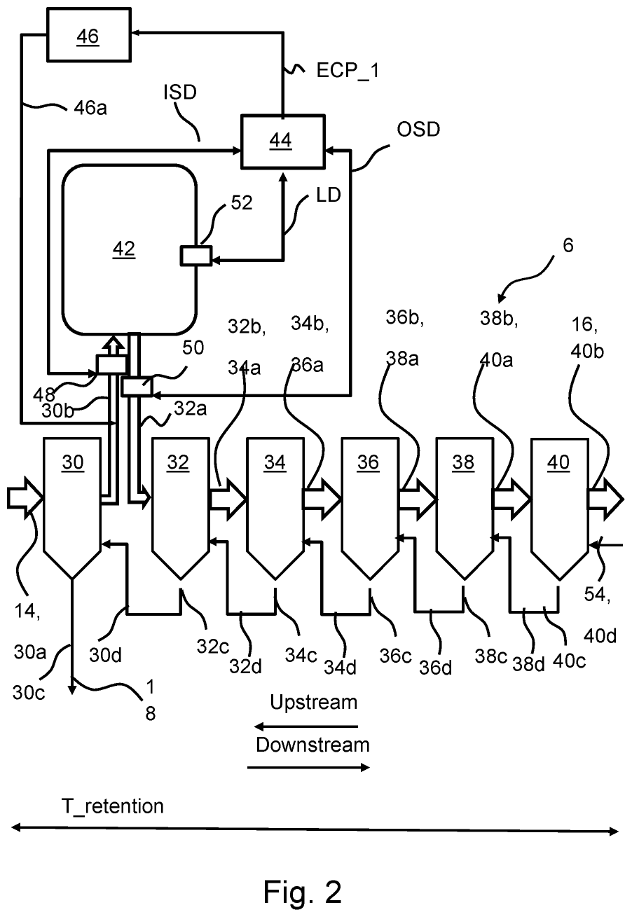

FIG. 1 shows an exemplary wet-milling system implementing the control system according to the present disclosure. The wet-milling system 2 comprises a preparation system 4, a fiberwashing system 6, 6A, a fiber slurry post-processing system 8, and a starch and protein extraction system 10. The control system 12 controls devices and operations in one or more of systems 4, 6/6A, 8, 10. The control system 12 may be a distributed control system with a central controller and one or more sub-controllers distributed in respective systems 4, 6/6A, 8, 10. The preparation system 4 typically includes one or more of steeping, destoning, dewatering, grinding, germ separation, and germ processing of the corn for provision of a slurry also denoted fiber slurry that is fed as fiber input 14 to the fiberwashing system 6. The fiberwashing system 6 washes the fiber slurry for provision of a fiber output 16 to the fiber output post-processing system 8 and for provision of a starch and protein output 18 to the starch and protein extraction system 10. The fiberwashing system 6 will be described in further detail in relation to FIG. 2. The fiber output post-processing system 8 typically includes one or more of pressing, mixing, and drying the slurry from fiber output 16 for provision of a fiber product, and the starch and protein extraction system 10 typically includes one or more of degritting, straining, centrifuging, washing, thickening, dewatering, and drying for provision of starch product 22 and/or protein or gluten product 24 based on the starch and protein output 18. It is noted that the terms slurry and fiber slurry are used interchangeably herein.

FIG. 2 shows a more detailed block diagram of an example fiberwashing system according to the present disclosure. The fiberwashing system 6 comprises a plurality of screening units including a first screening unit 30 and a second screening unit 32 downstream the first screening unit. Further, third screening unit 34, fourth screening unit 36, fifth screening unit 38, and sixth screening unit 40 are provided downstream the second screening unit 32. The number and positioning of screening units may be adjusted to different applications and specifications but is typically in the range from 4 to 10. Fiber slurry 14 from the preparation system 4 is fed as first fiber input 30a to the first screen unit 30 arranged in a first stage of the fiberwashing system. The fiber slurry 14 is washed in the first screening unit 30 for provision of a first fiber output 30b and a first wash output 30c, the first fiber output 30b forming a part of the process stream, and the first wash output 30c forming part of the milled starch stream. The first wash output 30c is fed as least as a part of the starch and protein output 18. The second screening unit 32 is arranged in a second stage of the fiberwashing system 6 and a second wash output 32c from the second screening unit 32 is fed as at least part of a first wash input 30d to the first screening unit 30.

The fiberwashing system 6 comprises an incubation tank 42. The first fiber output 30b is slurry or fiber slurry from the first screening unit 30 and is fed as fiber input to the incubation tank 42, and a fiber output of the incubation tank is fed as second fiber input 32a to the second screening unit 32. In other words, the incubation tank 42 is arranged between first fiber output 30b and second fiber input 32a.

A control system for the fiberwashing system is provided, the control system comprising a controller 44 comprising one or more processors and an interface. The control system comprises one or more sensor devices wired or wirelessly connected to the interface of the controller 44.

The fiberwashing system comprises a first enzyme input device 46 configured to feed a first enzyme or first enzyme composition 46a into the incubation tank 42. The first enzyme input device 46 is connected to the controller 44 for control of the first enzyme input device 46 by the controller 44.

The fiberwashing system 6 comprises an input sensor device 48 arranged at the input of the incubation tank 42. The input sensor device 48 is connected to the controller 44 for provision of input sensor data ISD to the controller 44. The input sensor device 48 may comprise a spectrometer and/or a density meter.

The fiberwashing system 6 optionally comprises an output sensor device 50 arranged at the output of the incubation tank 42. The output sensor device 50 is connected to the controller 44 for provision of output sensor data OSD to the controller 44.

The fiberwashing system 6 optionally comprises a tank sensor device including a level sensor device 52 arranged in and/or at the incubation tank 42. The level sensor device 52 is connected to the controller 44 for provision of level data LD to the controller 44, the level data LD indicative of slurry level/volume in the incubation tank 42.

The one or more processors of the controller 44 are configured to obtain fiber data comprising one or more input fiber parameters indicative of fiber input to the incubation tank 42. The fiber data comprise and/or are based on, such as determined as a function of, the input sensor data ISD from the input sensor device 48.

The one or more processors of the controller 44 are configured to obtain a time parameter indicative of an incubation time for slurry in the incubation tank. The time parameter is optionally based on, such as determined as a function of, the level data LD from level sensor device 52. The time parameter is optionally based on, such as determined as a function of, the input sensor data ISD from the input sensor device 48 and/or the output sensor data OSD from the output sensor device 50.

The one or more processors of the controller 44 are configured to determine an input scheme based on the fiber data and the time parameter, the input scheme comprising and/or defining an enzyme dosing scheme based on the fiber data and the time parameter. The enzyme dosing scheme comprises a first enzyme flowrate for a first enzyme. The controller 44/one or more processors transmits a first enzyme control parameter ECP_1 representative of the first enzyme flowrate as a control signal to the first enzyme input device 46 to control the first enzyme input device 46 according to the enzyme dosing scheme. In other words, the controller 44 is configured to control one or more input devices of the wet milling system according to the input scheme.

The input sensor device 48 is arranged at the input of the incubation tank 42 and configured to sense or measure one or more properties of the fiber input to the incubation tank 42. In other words, the input sensor data ISD are indicative of one or more properties, such as weight, density, flow speed, of the fiber or fiber slurry passing the input sensor device 48. Thus, the input sensor device 48 may comprise a weight meter also denoted a mass meter.

The input sensor device 48 may comprise a spectrometer optionally comprising one or more MIR and/or one or more NIR sensors for provision of spectrometer data of the fiber input as part of the input sensor data ISD.

The output sensor device 50 is arranged at the output of the incubation tank 42 and configured to sense or measure one or more properties of the fiber output from the incubation tank 42. In other words, the output sensor data OSD are indicative of one or more properties, such as weight, density, flow speed, of the fiber or fiber slurry passing the output sensor device 50. Thus, the output sensor device 50 may comprise a weight meter also denoted a mass meter.

The output sensor device 50 may comprise a spectrometer optionally comprising one or more MIR and/or one or more NIR sensors for provision of spectrometer data of the fiber output as part of the output sensor data OSD.

The sixth wash input 40d is a process water input 54 and is fed with process water.

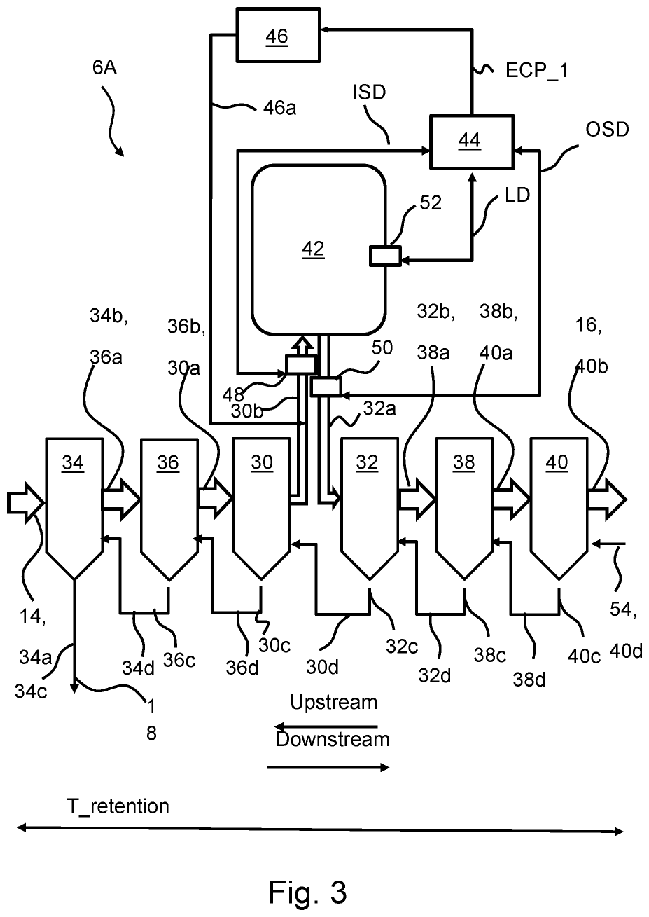

FIG. 3 shows a more detailed block diagram of an example fiberwashing system according to the present disclosure. The fiberwashing system 6A comprises a plurality of screening units including a first screening unit 30 and a second screening unit 32 downstream the first screening unit. Further, third screening unit 34 and fourth screening unit 36 are provided upstream the first screening unit 30. The third fiber input 34a of the third screening unit 34 is configured as the fiber input 14 of the fiberwashing system 6A. A fifth screening unit 38, and sixth screening unit 40 are provided downstream the second screening unit 32.

In fiberwashing system 6A, the incubation tank 42 is arranged between intermediate stages, namely between the third stage and the fourth stage, i.e. between the first screening unit 30 arranged in the third stage and the second screening unit arranged in the fourth stage of the fiberwashing system. The first fiber output 30b is slurry or fiber slurry from the first screening unit 30 and is fed as fiber input to the incubation tank 42, and a fiber output of the incubation tank is fed as second fiber input 32a to the second screening unit 32. In other words, the incubation tank 42 is arranged between first fiber output 30b and second fiber input 32a.

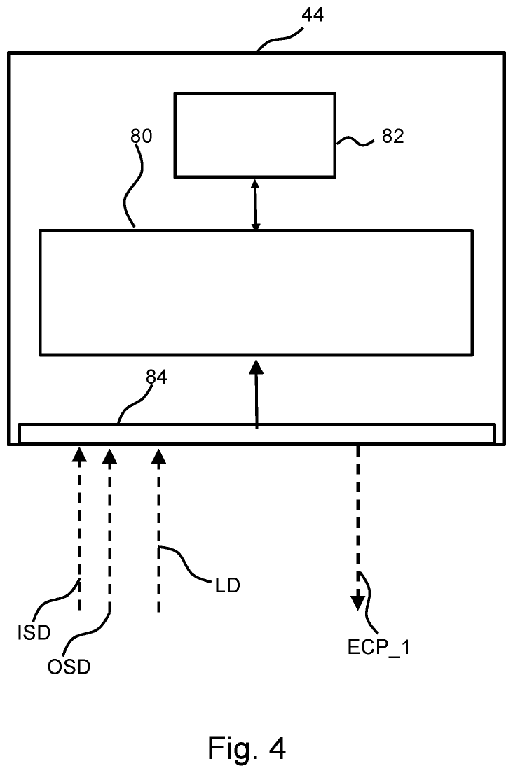

FIG. 4 shows an exemplary controller of a control system according to the present disclosure. The controller 44 comprises one or more processors 80, memory 82 and an interface 84. The interface 84 connects (wired and/or wirelessly) the controller 44 to sensor device(s), such as sensor devices 48, 50, 52 and/or input device(s), such as input device 46, of the fiberwashing system. The one or more processors 80 are connected to memory 82, the memory 82 storing input scheme and/or system configuration parameters or other settings relevant for the operation of controller 44.

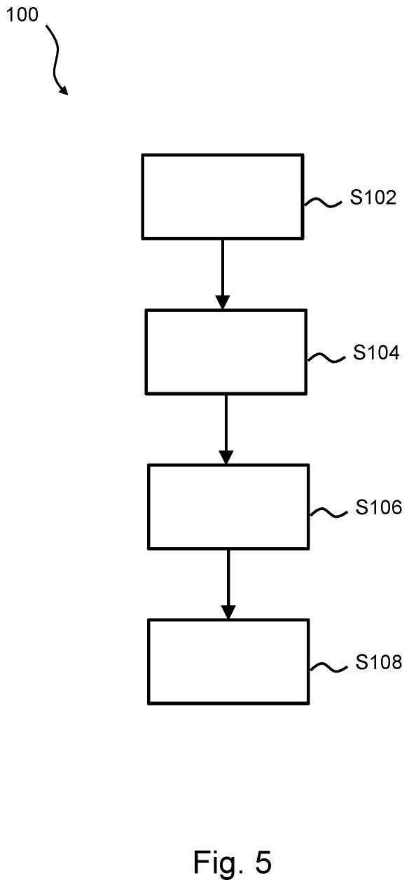

FIG. 5 is a flow chart of an exemplary method of producing a fiber output. The method 100 is a computer-implemented method of producing a fiber output from a fiber washing system of a wet milling system, the method 100 comprising obtaining S102 fiber data comprising an input fiber parameter indicative of a fiber input to an incubation tank of the fiber washing system; obtaining S104 a time parameter indicative of an incubation time for slurry in the incubation tank; determining S106 an input scheme based on the fiber data and the time parameter; and controlling S108 one or more input devices of the wet milling system according to the input scheme.

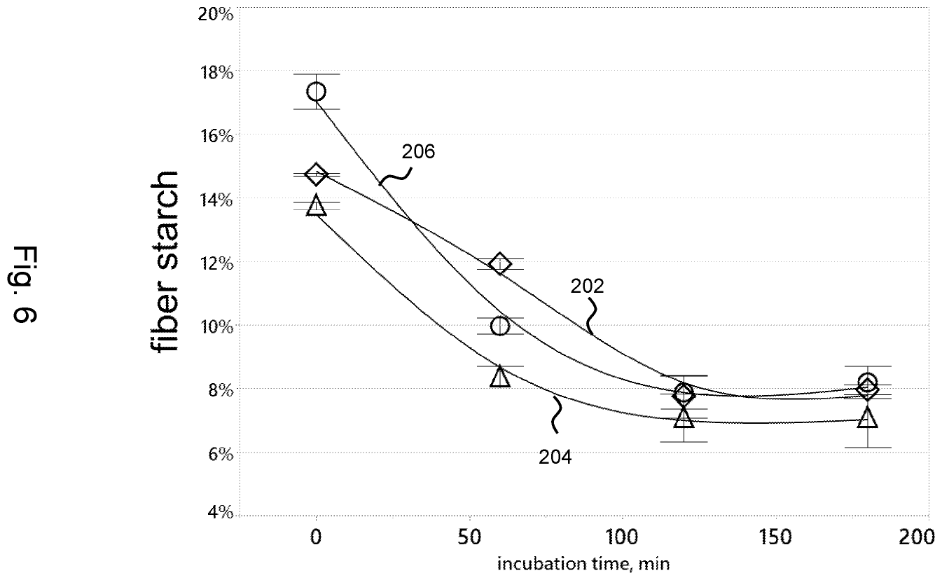

FIG. 6 shows example starch release profiles (bound starch vs fiber solids) for an enzyme composition (xylanase A and cellulase A) for different combinations of fiber content and enzyme in fiber slurry. The release profiles are measured in a stationary setting but may be transformed to include or take into account time for converting to flow. The curve 202 (diamonds) illustrates starch release as a function of incubation time for a fiber solid content of 3% and an amount of 706 microgram enzyme protein/g dry fiber solids in 1.2 kg of fiber slurry. The curve 204 (triangles) illustrates starch release as a function of incubation time for a fiber solid content of 4.5% and an amount of 706 microgram enzyme protein/g dry fiber in 1.2 kg of fiber slurry. The curve 206 (circles) illustrates starch release as a function of incubation time for a fiber solid content of 6% and an amount of 1.412 milligram enzyme protein/g dry fiber solids in 1.2 kg of fiber slurry. The starch release profiles 202, 204, 206 were obtained with temperature setting at 48 C, pH at 4.2 to 4.5, and agitation at 200 rpm with a cellulase and xylanase cocktail as the enzyme.

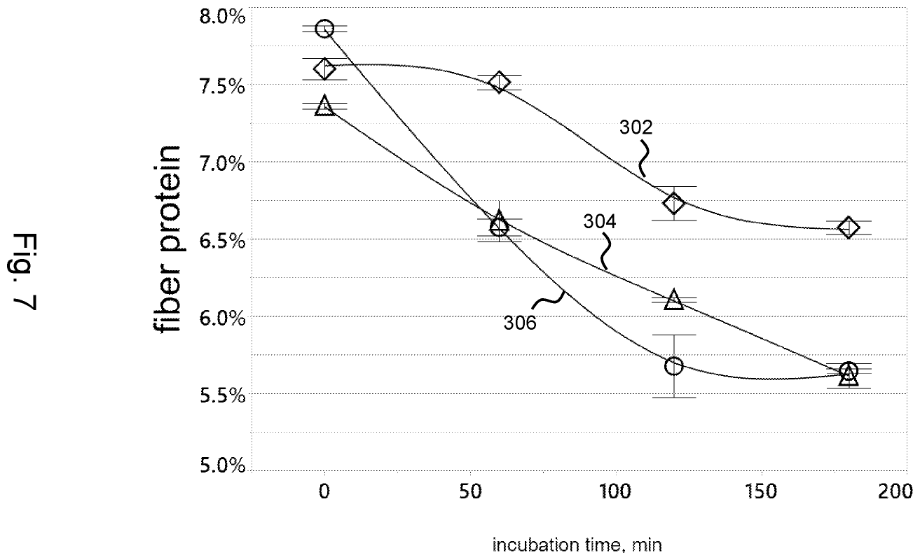

FIG. 7 shows example protein release profiles (bound protein vs fiber solids) for an enzyme composition (xylanase A and cellulase A) for different combinations of fiber content and enzyme in the fiber slurry. The release profiles are measured in a stationary setting but may be transformed to include or take into account time. The curve 302 (diamonds) illustrates protein release as a function of incubation time for a fiber solid content of 3% and an amount of 706 microgram enzyme protein/g dry fiber in 1.2 kg of fiber slurry. The curve 304 (triangles) illustrates protein release as a function of incubation time for a fiber solid content of 4.5% and an amount of 706 microgram enzyme protein/g dry fiber in 1.2 kg of fiber slurry. The curve 306 (circles) illustrates protein release as a function of incubation time for a fiber solid content of 6% and an amount of 1.412 milligram enzyme protein/g dry fiber solids in 1.2 kg of fiber slurry. The protein release profiles 302, 304, 306 were obtained with temperature setting at 48 C, pH at 4.2 to 4.5, and agitation at 200 rpm with a cellulase and xylanase cocktail as the enzyme.

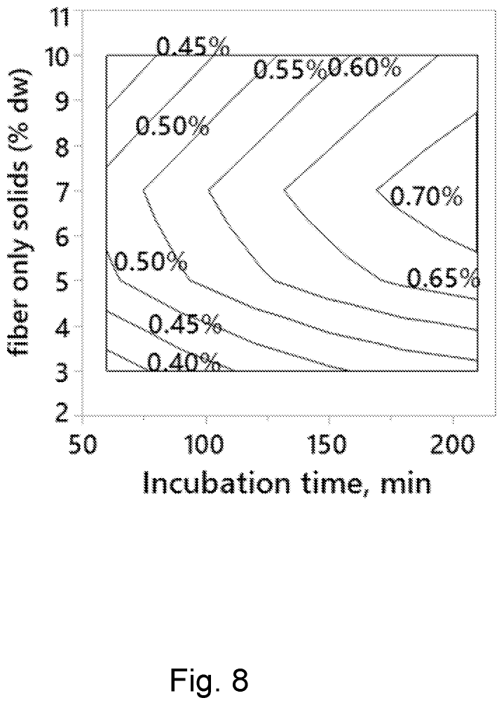

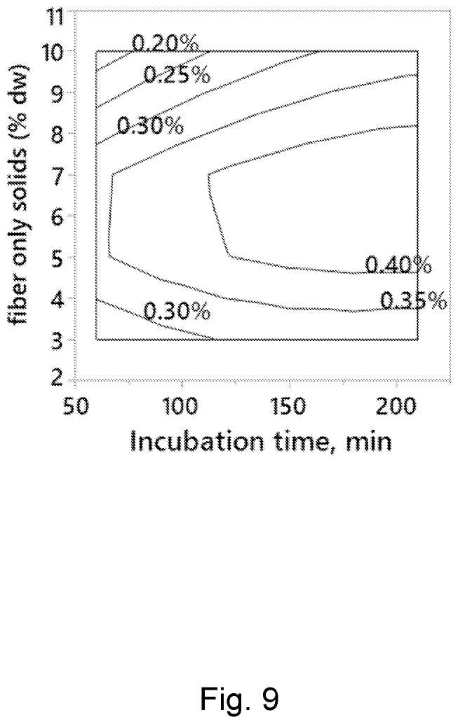

FIG. 8 and FIG. 9 illustrates an enzyme performance model for protein yield (contour lines) across incubation time and fiber solids for different enzyme doses of 0.4 kg enzyme product/ton corn and 0.1 kg enzyme product/ton corn, respectively. FIG. 8 and FIG. 9 show different ranges of protein yields across fiber solids and incubation time. Based on distinction of protein yield contour line responses in FIG. 8 and FIG. 9, an enzyme dose model is applicable to optimize the target protein yields for a corn wet-milling plant by controlling the enzyme dose based on fiber solid content and incubation time.

The use of the terms “first”, “second”, “third” and “fourth”, “primary”, “secondary”, “tertiary” etc. does not imply any particular order but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Memory may be one or more of a buffer, a flash memory, a hard drive, a removable media, a volatile memory, a non-volatile memory, a random access memory (RAM), or other suitable device. In a typical arrangement, memory may include a non-volatile memory for long term data storage and a volatile memory that functions as system memory for the processor. Memory may exchange data with processor over a data bus. Memory may be considered a non-transitory computer readable medium.

Memory may be configured to store information (such as information indicative of the parameters and/or models of the feed control) in a part of the memory.

Furthermore, the labelling of a first element does not imply the presence of a second element and vice versa.

It may be appreciated that FIGS. 1-9 comprise some modules or operations which are illustrated with a solid line and some modules or operations which are illustrated with a dashed line. The modules or operations which are comprised in a solid line are modules or operations which are comprised in the broadest example embodiment. The modules or operations which are comprised in a dashed line are example embodiments which may be comprised in, or a part of, or are further modules or operations which may be taken in addition to the modules or operations of the solid line example embodiments. It should be appreciated that these operations need not be performed in order presented. Furthermore, it should be appreciated that not all of the operations need to be performed. The exemplary operations may be performed in any order and in any combination.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the exemplary embodiments may be implemented at least in part by means of both hardware and software, and that several “means”, “units” or “devices” may be represented by the same item of hardware.

The various exemplary methods, devices, and systems described herein are described in the general context of method steps processes, which may be implemented in one aspect by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform specified tasks or implement specific abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

### LIST OF REFERENCES

2 wet-milling system
4 preparation system
6, 6A fiberwashing system
8 fiber output post-processing system
10 starch and protein extraction system
12 control system
14 fiber input, fiber slurry, slurry
16 fiber output
18 starch and protein output
20 fiber product
22 starch product
24 protein/gluten product
30 first screening unit
30a first fiber input
30b first fiber output
30c first wash output
30d first wash input
32 second screening unit
32a second fiber input
32b second fiber output
32c second wash output
32d second wash input
34 third screening unit
34a third fiber input
34b third fiber output
34c third wash output
34d third wash input
36 fourth screening unit
36a fourth fiber input
36b fourth fiber output
36c fourth wash output
36d fourth wash input
38 fifth screening unit
38a fifth fiber input
38b fifth fiber output
38c fifth wash output
38d fifth wash input
40 sixth screening unit
40a sixth fiber input
40b sixth fiber output
40c sixth wash output
40d sixth wash input
42 incubation tank
44 controller
46 first enzyme input device
46a first enzyme, first enzyme composition
48 input sensor device
50 output sensor device
52 level sensor device
54 process water input
80 one or more processors
82 memory
84 interface
100 computer-implemented method of producing a fiber output
S102 obtaining fiber data comprising an input fiber parameter indicative of a fiber input to an incubation tank of the fiber washing system
S104 obtaining a time parameter indicative of an incubation time for slurry in the incubation tank
S106 determining an input scheme based on the fiber data and the time parameter
S108 controlling one or more input devices
202 starch release curve
204 starch release curve
206 starch release curve
302 protein release curve
304 protein release curve
306 protein release curve

The present invention is further disclosed in the following numbered embodiments.

Embodiment 1. A control system for a fiber washing system of a wet milling system, the control system comprising a controller comprising one or more processors and an interface, wherein the one or more processors are configured to:
obtain fiber data comprising an input fiber parameter indicative of a fiber input to an incubation tank of the fiber washing system;
obtain a time parameter indicative of an incubation time for slurry in the incubation tank;
determine an input scheme based on the fiber data and the time parameter; and
control one or more input devices of the wet milling system according to the input scheme.

Embodiment 2. Control system according to embodiment 1, wherein the input fiber parameter is indicative of a fiber content in a slurry input to the incubation tank.

Embodiment 3. Control system according to any one of embodiments 1-2, wherein to obtain a time parameter indicative of an incubation time for slurry in the incubation tank comprises to obtain a tank level parameter and to determine the time parameter based on the tank level parameter.

Embodiment 4. Control system according to any one of embodiments 1-3, wherein to obtain a time parameter indicative of an incubation time for slurry in the incubation tank comprises to obtain a tank input flow parameter and to determine the time parameter based on the tank input flow parameter.

Embodiment 5. Control system according to any one of embodiments 1-4, wherein to obtain a time parameter indicative of an incubation time for slurry in the incubation tank comprises to obtain a tank output flow parameter and to determine the time parameter based on the tank output flow parameter.

Embodiment 6. Control system according to any one of embodiments 1-5, wherein the fiber data comprises a constituent input parameter indicative of a quantity of insoluble protein and starch entering the incubation tank, and wherein to determine an input scheme based on the fiber data and the time parameter comprises to determine the input scheme based on the constituent input parameter.

Embodiment 7. Control system according to any one of embodiments 1-6, wherein the fiber data comprises a protein input parameter indicative of a quantity of insoluble protein entering the incubation tank, and wherein to determine an input scheme based on the fiber data and the time parameter comprises to determine the input scheme based on the protein input parameter.

Embodiment 8. Control system according to any one of embodiments 1-7, wherein the fiber data comprises a starch input parameter indicative of a quantity of insoluble starch entering the incubation tank, and wherein to determine an input scheme based on the fiber data and the time parameter comprises to determine the input scheme based on the starch input parameter.

Embodiment 9. Control system according to any one of embodiments 1-8, wherein the one or more processors are configured to obtain a protein output parameter indicative of a quantity of insoluble protein exiting the incubation tank, and wherein to determine an input scheme based on the fiber data and the time parameter comprises to determine the input scheme based on the protein output parameter.

Embodiment 10. Control system according to any one of embodiments 1-9, wherein to determine an input scheme comprises to determine an enzyme dosing scheme based on the fiber data and the time parameter, the enzyme dosing scheme comprising a first enzyme flowrate for a first enzyme, and wherein to control one or more input devices comprises to control a first enzyme input device according to the enzyme dosing scheme.

Embodiment 11. Control system according to embodiment 10, wherein to control an input device comprises to control the first enzyme input device according to the first enzyme flowrate.

Embodiment 12. Control system according to any of embodiments 10-11, wherein the enzyme dosing scheme comprises a second enzyme flowrate for a second enzyme, and wherein to control an input device comprises to control a second enzyme input device according to the second enzyme flowrate.

Embodiment 13. Control system according to any of embodiments 10-12, wherein the first enzyme is selected from a xylanase and a cellulase or is a first enzyme composition comprising one or more of a xylanase and a cellulase.

Embodiment 14. Control system according to embodiment 13, wherein the xylanase belongs to the GH family 5, 8, 10, 11, or 30 and comprises one or more of a GH5 xylanase, GH8 xylanase, GH30 xylanase, GH10 xylanase, and GH11 xylanase.

Embodiment 15. Control system according to any of embodiments 13-14, wherein the cellulase comprises one or more of endoglucanase, cellobiohydrolase, and beta-glucosidase.

Embodiment 16. Control system according to any of embodiments 1-15, wherein to determine an input scheme comprises to determine a feed rate, and wherein to control one or more input devices comprises to control a feed input device according to the feed rate.

Embodiment 17. Control system according to any of embodiments 1-16, wherein the fiber data comprises spectrometer data of the fiber input, and wherein to obtain fiber data comprises to determine a first component parameter of a first component of the fiber input based on the spectrometer data.

Embodiment 18. Control system according to embodiment 17, wherein the first component is one of starch, protein, and fiber.

Embodiment 19. Control system according to any of embodiments 1-18, wherein the fiber data comprises temperature data indicative of a temperature of the fiber input, and wherein to determine an input scheme based on the fiber data and the time parameter comprises to determine the input scheme based on the temperature data.

Embodiment 20. Control system according to any of embodiments 1-19, wherein the fiber data comprises pH data indicative of a pH of the fiber input, and wherein to determine an input scheme based on the fiber data and the time parameter comprises to determine the input scheme based on the pH data.

Embodiment 21. Fiber washing system comprising a control system according to any of embodiments 1-20, wherein the fiber washing system comprises an incubation tank and a plurality of screening units including a first screening unit and a second screening unit downstream the first screening unit, the first screening unit having a first fiber input and a first fiber output, and the second screening unit having a second fiber input and a second fiber output, wherein a first fiber output of the first screening unit or a second fiber output of the second screening unit is coupled to an incubation input of the incubation tank.

Embodiment 22. An enzymatic corn wet-milling process comprising using the control system according to any of embodiments 1-20 for fiber washing in the wet-milling process.

Embodiment 23. The enzymatic corn wet-milling process of embodiment 22, wherein starch yield and/or gluten yield from ground corn kernels is increased compared to an enzymatic corn wet-milling process where no control system is in place.

Embodiment 24. The enzymatic corn wet-milling process of any of embodiments 22-23, wherein the enzymatic corn wet-milling process comprises:
a) soaking the corn kernels in water to produce soaked kernels;
b) grinding the soaked kernels to produce ground kernels;
c) separating germ from the ground kernels to produce a corn kernel mass comprising fiber, starch and gluten; and
d) subjecting the corn kernel mass to a fiber washing procedure separating starch and gluten from the fiber to produce a fiber output,
wherein at least one xylanase are present/added before or during step d), and wherein the control system is applied to an enzyme incubation tank in the fiber-washing system in step d).

Embodiment 25. The enzymatic corn wet-milling process of embodiment 24 comprising:
e) separating the starch from the gluten; and
f) washing the starch.

Embodiment 26. A use of a control system according to any of embodiments 1-20 in a corn wet-milling process.

Embodiment 27. The use according to embodiment 26, wherein the control system is applied to an enzyme incubation tank integrated into a fiber-washing system.

Embodiment 28. A computer-implemented method of producing a fiber output from a fiber washing system of a wet milling system, the method comprising:
obtaining fiber data comprising an input fiber parameter indicative of a fiber input to an incubation tank of the fiber washing system;
obtaining a time parameter indicative of an incubation time for slurry in the incubation tank;
determining an input scheme based on the fiber data and the time parameter; and
controlling one or more input devices of the wet milling system according to the input scheme.

The invention is further illustrated in more detail in the examples below.

### EXAMPLES

### Example 1. Improved Wet-Milling Process Including Control System According to the Invention

The enzymatic Fiber-washing step in corn wet-milling processes utilize application of single or blends of enzyme products to increase starch and protein yields. The fiber slurry is mixed with enzymes (cellulases: exo/endo glucanases, cellobiohydrolase, beta-glucosidase; hemicellulases, e.g., GH family 5, 8, 10, 11, or 30 xylanases) in an incubation tank for certain required amount of time, which is also referred to as incubation time.

The enzymes aid in releasing fiber bound insoluble starch and protein, which ultimately increase starch and protein yields in the overall wet-milling process. These yield results are dependent on incoming fiber parameters (for example, fiber solids, bound starch and protein values), incubation time, temperature, pH and most importantly dosage of enzyme. While incubation time, temperature and pH values are routinely monitored and adjusted in plant operation, the enzyme dosing is not and is based on volume of corn kernel being steeped and ground in initial stages of the process, which precedes the enzymatic fiber-washing step by 24 to 48 hours. Another input parameter—fiber solid is not monitored and has huge fluctuations in the fiber slurry heading into incubation tank. Therefore, the current process has disparity in adequate enzyme dose to fiber solids directed to incubation tank. This practice impacts enzyme efficiency and leads to major variation in quantities of starch and protein yields specifically due to over or under dosing of enzymes required to keep consistent yield targets.

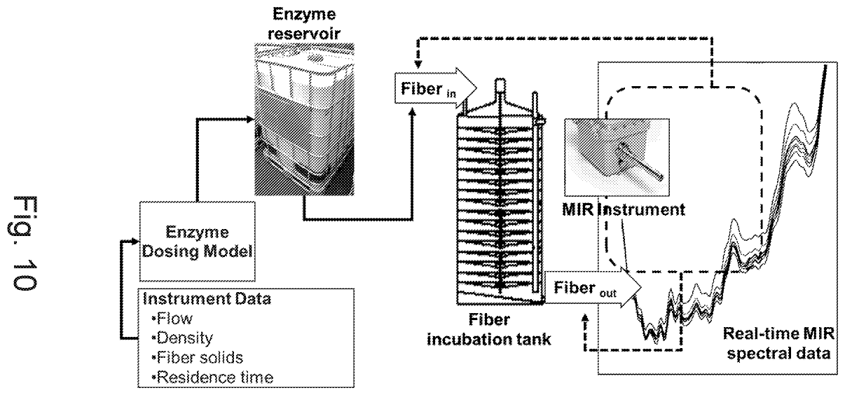

As shown in FIG. 10, a control system for a fiber-washing system can be developed to determine input scheme (e.g., enzyme dosing) by collecting real-time incubation tank input parameters (i.e., fiber flow, density, solids, temperature, pH and spectrometer readings) and output parameters (i.e., fiber flow, density, solids, temperature, pH and spectrometer readings). For example, a typical corn wet-milling industry plant processes 800 to 1250 tons of corn per day. The bulk volume of corn is steeped for 24 hours, followed by first grind to separate germ—oil bearing tissues, then second and third grind to release and separate starch and protein present in mill stream via series of typically 4 to 8, e.g., six fiber-washing screening units. In the enzymatic fiber-washing process, a fiber slurry stream is diverted, after 2^{nd} fiber-washing station/screening unit, to an incubation tank, where the enzyme product reacts with the fiber slurry for certain time (e.g., 150 minutes). During this time, the enzyme enhances release of bound starch and protein in fiber. After incubation, the fiber slurry is directed to remaining series of fiber-washing stations/screening units, where counter current wash water aids to wash and separate starch and protein from bulk fiber slurry. The amount of fiber solids heading into incubation tank can be determined using mass flow and density meter installed at the inlet of the tank. Fiber solids entering to incubation tank typically vary between 3 to 6%. A mid infrared (MIR) probe/sensor (for example, KEIT MIR probe) installed at the inlet and outlet of the incubation tank records changes in fiber hydrolysis analyte profiles. Temperature and pH probes can also be installed at the inlet and outlet. Together, data collected by these instruments are referred to as input and outlet fiber parameters. The MIR spectral data at inlet and outlet of incubation tank are tied to performance of enzyme composition. For optimal performance, enzyme dosing is adjusted based on calibrated input and output fiber parameters, for example: fiber solids, MIR spectra profiles, starch and protein in fiber.

### Example 2. Correlation Between Fiber Concentration in Slurry, Enzyme Dose and Incubation Time in Tank

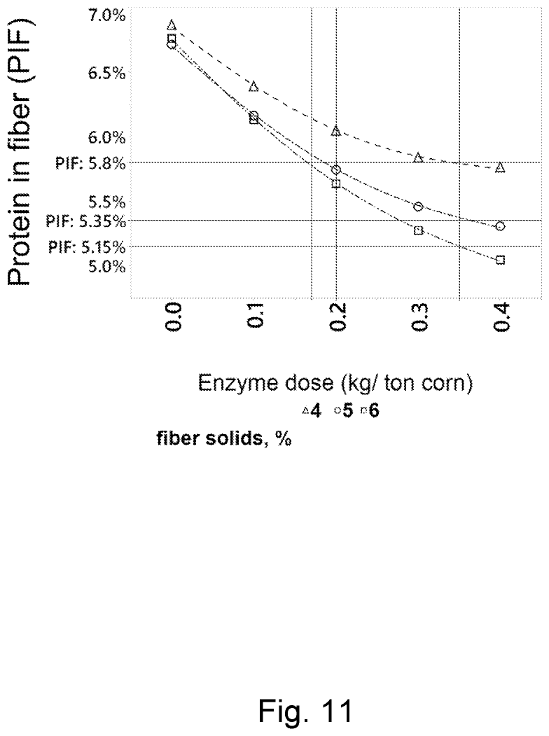

The Fiber-washing incubation system will receive fiber slurries at various fiber solid concentrations. For example, the fiber solid concentration can vary between 4 to 6%. Fixed enzyme dosing to the enzyme incubation tank can lead to different level of protein yield performance. An enzyme or mix of enzymes (for example, endoglucanase, cellobiohydrolase, beta-glucosidase, beta-xylosidase, arabinofuranosidase and xylanase) can be used to improve insoluble protein recovery. In such enzymatic fiber-washing process, reduction of protein in fiber (PIF) is an indication of enzyme-assisted recovery of proteins from fiber. As exemplified in FIG. 11, enzyme dose can be determined real-time to achieve target protein recovery if other input parameters, for example: fiber flow & solids, can be retrievable at the same time. The example illustrates the performance of an enzyme composition containing Trichoderma reesei cellulases and a GH5 xylanase at various doses versus various fiber solid contents. The total incubation time was 150 minutes, reaction pH and temperature were respectively 4.4 and 48 degree Celsius. The enzyme dose performance is dependent on fiber solids concentration. As illustrated, at same dose, for example: 0.35 kg enzyme/ton corn, the level of performance varied across fiber solids range. Here, the protein in fiber (PIF) was reduced to 5.8%, 5.35% and 5.15% respectively when fiber solid was 4%, 5% and 6%.

To maintain consistent enzyme performance across varying range of fiber solids, the optimal dose of enzyme can be adjusted depending on input parameter, for example: fiber solids. As described in FIG. 11, to achieve a certain level of residual protein in fiber, for example: 5.8%, the enzyme dosing was adjusted to 0.35, 0.2 and 0.17 kg/ton corn respectively for 4%, 5% and 6% fiber solids heading into reaction vessel, which is also referred to as incubation tank. Thereby, the enzyme dose was optimized for fiber solids.

### Example 3. Real Time Monitoring of Enzyme Hydrolysis of Fiber Slurry and Release of Bound Starch and Protein

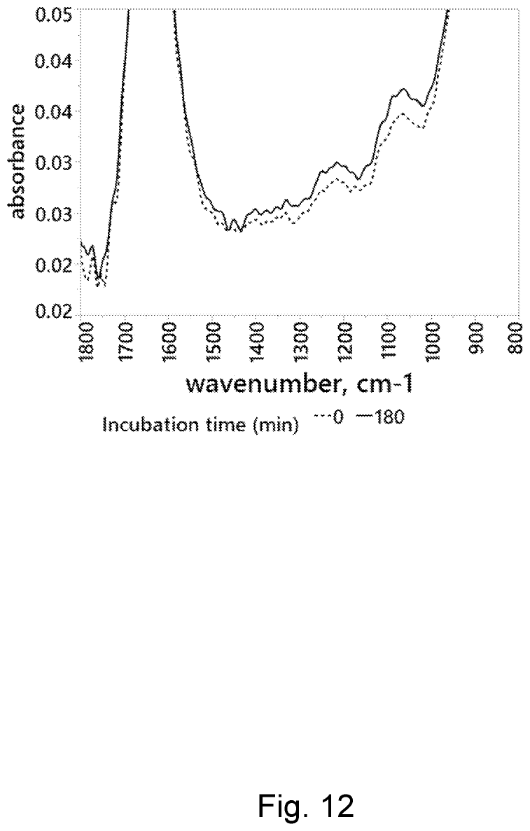

In-line spectrometer data was collected using Mid Infrared (MIR) probes/sensors, for example: KEIT MIR probes, installed at the inlet and outlet of incubation tank, where corn fiber is reacted with enzyme(s). As shown in FIG. 12, the fiber hydrolysis activity of enzyme can be distinguished between two time points via spectrometer data recorded by MIR probes installed at the inlet and outlet of the tank. The inlet MIR spectral data was referenced as T=0, while the outlet MIR spectral data was referenced as T=180 minutes. During that time, i.e., 180 minutes, enzyme product, for example: cellulase and xylanase blend, hydrolysed corn fiber to release bound starch and protein. The levels of fiber hydrolysis were assessed based on the model, which comprises of trained input (i.e., T=0) parameter data and output (T=180 minutes) parameter data. These parameters were input fiber solids, protein in fiber, starch in fiber, in-line process data, spectral data as well as output spectral data, protein in fiber, starch in fiber.

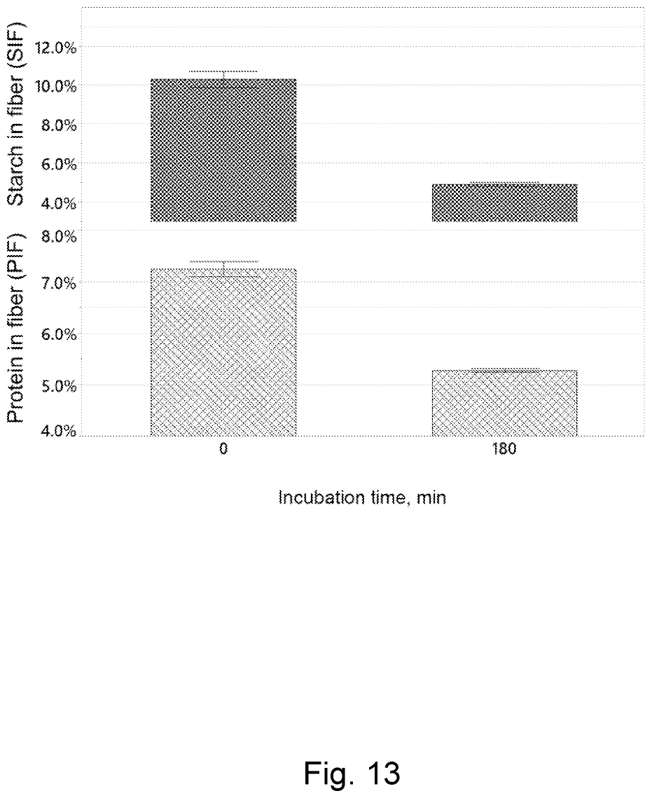

The example, provided in FIG. 12, represents collection of MIR spectral data at T=0 minutes and T=180 minutes for hydrolysis of 4.5% corn fiber solids with enzyme blend (cellulases and GH5 xylanase) dosed at 0.25 kg/ton corn, pH 4.4 and temperature 48 degree Celsius. Reduction of residual starch in fiber (SIF), 10.3% to 4.9%, and protein in fiber (PIF), 7.2% to 5.3%, values was recorded at T=180 minutes, as shown in FIG. 13. Therefore, the enzymatic Fiber-washing process aided in recovery of extra starch and protein from corn fiber. The enzyme shows similar performance at commercial scale operation. The spectral MIR data, in FIG. 12, represent the real-time changes in spectral profiles of the enzymatic corn fiber hydrolysis reaction. Such in-line MIR data can be used in developing control system to manage dosing and maintaining optimal performance of fiber hydrolyzing enzymes at commercial scale.

### Example 4. Sugar Analytes Released by Enzymatic Action on Fiber During Fiber-Wash

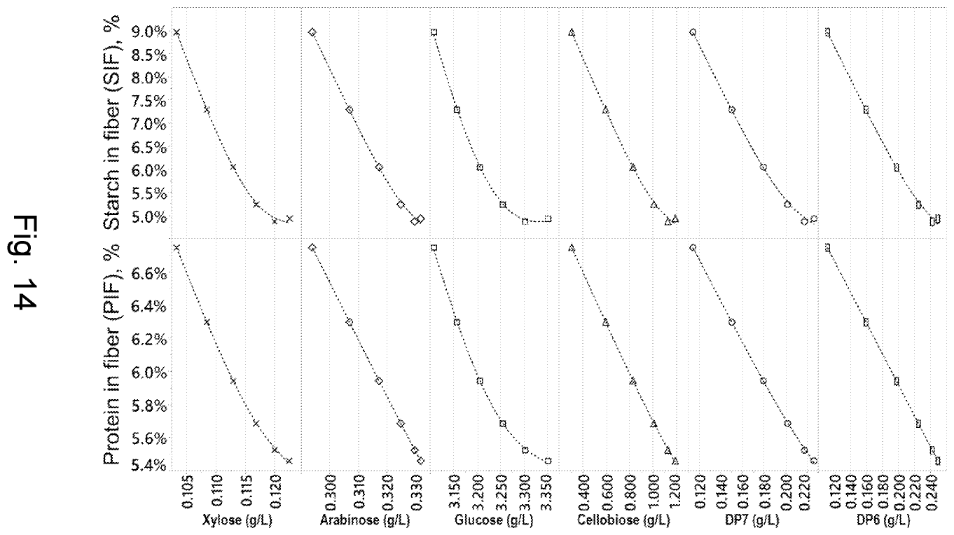

The enzymatic hydrolysis process release five and six carbon sugar molecules. These can be monomer sugar (DP1) products: xylose, arabinose, and glucose; disaccharide (DP2): cellobiose; and oligosaccharides: DP6 and DP7. The plots in FIG. 14 show incremental release of these DP1, DP2, DP6 or DP7 sugars, along incubation time (0 to 150 minutes), vs residual protein or starch in fiber. The example illustration was performed as follows: enzyme dosing: 0.3 kg Enzyme/ton corn, fiber solids: 5% and incubation time: 0 to 150 minutes at 48 degree Celsius and pH 4.4. As reaction (incubation) time progresses, the concentrations of various sugar molecules increased (left to right in horizontal—X axis) and release of fiber bound insoluble (recoverable) starch and protein increased—whereby, the residual starch in fiber (SIF) and protein in fiber (PIF) decreased (top to bottom, towards right direction, in vertical—Y axis). Changes of these analyte profiles in bulk liquid stream can be correlated with changes in MIR spectra (in 800 to 1800 cm⁻¹ region, as shown in FIG. 12) well as SIF and PIF data.

Although features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications, and equivalents.

1. A control system for a fiber washing system of a wet milling system, the control system comprising a controller comprising one or more processors and an interface, wherein the one or more processors are configured to:
obtain fiber data comprising an input fiber parameter indicative of a fiber input to an incubation tank of the fiber washing system;
obtain a time parameter indicative of an incubation time for slurry in the incubation tank;
determine an input scheme based on the fiber data and the time parameter; and
control one or more input devices of the wet milling system according to the input scheme; and
wherein the input fiber parameter is indicative of a fiber content in a slurry input to the incubation tank.

2. The control system according to claim 1, wherein to obtain a time parameter indicative of an incubation time for slurry in the incubation tank comprises to obtain a tank level parameter and to determine the time parameter based on the tank level parameter.

3. The control system according to claim 1, wherein to obtain a time parameter indicative of an incubation time for slurry in the incubation tank comprises to obtain a tank input flow parameter and to determine the time parameter based on the tank input flow parameter.

4. The control system according to claim 1, wherein to obtain a time parameter indicative of an incubation time for slurry in the incubation tank comprises to obtain a tank output flow parameter and to determine the time parameter based on the tank output flow parameter.

5. The control system according to claim 1, wherein the fiber data comprises a constituent input parameter indicative of a quantity of insoluble protein and starch entering the incubation tank, and wherein to determine an input scheme based on the fiber data and the time parameter comprises to determine the input scheme based on the constituent input parameter.

6. The control system according to claim 1, wherein the fiber data comprises a protein input parameter indicative of a quantity of insoluble protein entering the incubation tank, and wherein to determine an input scheme based on the fiber data and the time parameter comprises to determine the input scheme based on the protein input parameter.

7. The control system according to claim 1, wherein the fiber data comprises a starch input parameter indicative of a quantity of insoluble starch entering the incubation tank, and wherein to determine an input scheme based on the fiber data and the time parameter comprises to determine the input scheme based on the starch input parameter.

8. The control system according to claim 1, wherein the one or more processors are configured to obtain a protein output parameter indicative of a quantity of insoluble protein exiting the incubation tank, and wherein to determine an input scheme based on the fiber data and the time parameter comprises to determine the input scheme based on the protein output parameter.

9. The control system according to claim 1, wherein to determine an input scheme comprises to determine an enzyme dosing scheme based on the fiber data and the time parameter, the enzyme dosing scheme comprising a first enzyme flowrate for a first enzyme, and wherein to control one or more input devices comprises to control a first enzyme input device according to the enzyme dosing scheme.

10. The control system according to claim 9, wherein to control an input device comprises to control the first enzyme input device according to the first enzyme flowrate.

11. The control system according to claim 9, wherein the enzyme dosing scheme comprises a second enzyme flowrate for a second enzyme, and wherein to control an input device comprises to control a second enzyme input device according to the second enzyme flowrate.

12. The control system according to claim 9, wherein the first enzyme is selected from a xylanase and a cellulase or is a first enzyme composition comprising one or more of a xylanase and a cellulase.

13. The control system according to claim 1, wherein to determine an input scheme comprises to determine a feed rate, and wherein to control one or more input devices comprises to control a feed input device according to the feed rate.

14. The control system according to claim 1, wherein the fiber data comprises spectrometer data of the fiber input, and wherein to obtain fiber data comprises to determine a first component parameter of a first component of the fiber input based on the spectrometer data.

15. The control system according to claim 14, wherein the first component is one of starch, protein, and fiber.

16. A fiber washing system comprising a control system according to claim 1, wherein the fiber washing system comprises an incubation tank and a plurality of screening units including a first screening unit and a second screening unit downstream the first screening unit, the first screening unit having a first fiber input and a first fiber output, and the second screening unit having a second fiber input and a second fiber output, wherein a first fiber output of the first screening unit or a second fiber output of the second screening unit is coupled to an incubation input of the incubation tank.

17. An enzymatic corn wet-milling process comprising the control system according to claim 1 for fiber washing in the wet-milling process.

18. The enzymatic corn wet-milling process of claim 17, wherein starch yield and/or gluten yield from ground corn kernels is increased compared to an enzymatic corn wet-milling process where no control system is in place.

19. (canceled)

20. A computer-implemented method of producing a fiber output from a fiber washing system of a wet milling system, the method comprising:
obtaining fiber data comprising an input fiber parameter indicative of a fiber input to an incubation tank of the fiber washing system;
obtaining a time parameter indicative of an incubation time for slurry in the incubation tank;
determining an input scheme based on the fiber data and the time parameter; and
controlling one or more input devices of the wet milling system according to the input scheme.
